(19) United States
(12) Patent Application Publication
(10) Pub. No.: US 2003/0036741 A1
(43) Pub. Date: Feb. 20, 2003

(54) Textured airlaid materials

(75) Inventors: Rodney L. Abba, Oshkosh, WI (US); Robert J. Makolin, Neenah, WI (US); David J. Nickel, Neenah, WI (US); Charles W. Colman, Marietta, GA (US); Jeffrey Dean Lindsay, Appleton, WI (US); Fung-Jou Chen, Appleton, WI (US); Julie Marie Bednarz, Neenah, WI (US)

Correspondence Address: TIMOTHY A. CASSIDY, Dority & Manning, Attorneys at Law, P.A., P.O. Box 1449, Greenville, SC, 29602, US

(73) Assignee: Kimberly-Clark Worldwide, Inc.

(21) Appl. No.: 10/029,111

(22) Filed: Dec. 21, 2001

Related U.S. Application Data
(63) Continuation-in-part of application No. 09/684,039, filed on Oct. 6, 2000.
(63) Continuation-in-part of application No. 09/680,719, filed on Oct. 6, 2000.
(60) Provisional application No. 60/159,629, filed on Oct. 14, 1999.
(60) Provisional application No. 60/159,629, filed on Oct. 14, 1999.

(51) Int. Cl. A61F 13/15; A61F 13/20

(52) U.S. Cl. 604/385.101; 604/359; 604/360; 604/365; 604/366; 604/367; 604/368; 604/380; 604/382

(57) ABSTRACT

A textured airlaid web is disclosed. The textured web is formed on a three-dimensional fabric under sufficient force to cause the web to conform to the surface of the fabric. The textured web includes, on a minute scale, peak areas and valley areas. The peak areas and valley areas can improved the liquid handling properties of the web. For instance, webs can be produced having improved absorbency characteristics and/or wicking characteristics.

### BACKGROUND OF THE INVENTION

Absorbent articles and structures, such as absorbent pads, absorbent cores, and absorbent webs, have been formed by employing various techniques. The absorbent articles are typically incorporated into various absorbent products, such as diapers, feminine napkins and incontinence garments. In many applications, the absorbent articles are formed in an airlaying process.

Conventional airlaying processes typically include one or more forming chambers that are placed over a moving foraminous forming surface, such as a forming screen. Fibrous materials and/or particulate materials are introduced into the forming chamber and a vacuum source is employed to draw an airstream through the forming surface. The airstream deposits the fibers and/or particulate material onto the moving forming surface.

Once deposited onto the forming surface, a non-woven web is formed. Subsequently, the non-woven web can be bonded together. For example, the web can be bonded together using an adhesive and/or can be thermally bonded together.

In the past, flat non-woven sheets have been produced generally according to the above-described process. In other embodiments, the non-woven article has been shaped into a contoured batt or pad which locates more absorbent material or fibers in those areas which are subjected to higher levels of fluid loading.

In general depending upon their use, the airlaid articles should have good absorbency properties and/or wicking properties. For example, when used as an absorbent layer in a product, the airlaid web should quickly absorb fluids and, once absorbed, retain the fluids within the structure. In this regard, many attempts have been made to improve the absorbency characteristics of airlaid webs.

### SUMMARY OF THE INVENTION

The present invention is generally directed to further improvements to airlaid webs. More particularly, the present invention is directed to the formation of textured airlaid webs that can have improved absorbent properties and wicking properties. For example, textured airlaid webs made according to the present invention have a substantial increase in surface area in comparison to planar webs and therefore have more surface area for contact with liquids. Further, textured webs made according to the present invention can be made having localized density gradients, i.e. containing high density and low density areas. The high density areas can improve the absorption and liquid retaining characteristics of the web, while the low density areas can facilitate wicking of fluids off the surface of the web.

In one embodiment, the textured airlaid fibrous web is made from natural fibers, synthetic fibers, or mixtures thereof. The airlaid web is formed on a three-dimensional fabric under sufficient force to cause the web to conform to the surface of the fabric. The resulting textured surface that is formed into the web includes a repeating pattern of peak areas and valley areas. The height of the web, which refers to the distance between the lowest point on the web and the highest point on the web, can be at least 25% greater than the average caliper or thickness of the web, particularly at least 50% greater than the caliper of the web, and more particularly at least 100% greater than the caliper of the web.

According to the present invention, the airlaid textured web is bonded together after formation of the textured surface. By bonding the web together, the web becomes resistant to compression and fluid collapse. The web can be thermally bonded together by incorporating binder fibers into the web. Alternatively, the web can be bonded together by applying an adhesive to the surfaces of the web or can be bonded using both an adhesive and binder fibers.

As described above, the textured airlaid web includes a repeating pattern of peak areas and valley areas. In general, the web can include at least one peak per inch in one direction of the web, such as in the machine direction or the cross-machine direction. Particularly, the web can include at least 3 peaks per inch in one direction, more particularly at least 5 peaks per inch in one direction, and more particularly at least 10 peaks per inch in one direction. When the peak areas extend in more than one direction on the web, the peak areas can be present in an amount of at least 2 peaks per square inch, particularly at least 9 peaks per square inch, and more particularly at least 15 peaks per square inch.

The basis weight of webs made according to the present invention can be from about 40 gsm to about 1500 gsm or greater. The density of the webs can be from about 0.01 grams per cubic centimeter to about 0.3 grams per cubic centimeter. Due to the textured surface, the web includes a substantial amount of surface area. For instance, the web can have a surface area that is at least 25% greater than the surface area of a planar web at the same basis weight, and particularly 100% greater than the surface area of a planar web at the same basis weight.

In one embodiment, the textured airlaid web can be constructed such that the peak areas have a higher density than the valley areas. For example, the density of the peak areas can be at least 25% greater than the density of the valley areas.

If desired, the textured airlaid web can be contoured or otherwise molded to have a particular shape. The textured web can be incorporated into various absorbent articles and products, such as diapers, feminine hygiene products, adult incontinent products, wiping products and the like.

In order to form textured airlaid webs in accordance with the present invention, the process includes the steps of combining fibrous materials with a gas and/or a mixture of gases. The gas and fiber mixture is directed onto a moving conveyer to form a non-woven web. The web is contacted with a fabric having a three-dimensional surface under sufficient force to cause the web to conform to the three-dimensional surface and thereby form a textured surface on the web. The textured surface includes a repeating pattern of peak areas and valley areas that correspond to the three-dimensional pattern present on the fabric. After the textured surface is formed, the airlaid web is bonded together by thermal bonding and/or through the use of an adhesive.

In general, the textured surface can be produced on the non-woven airlaid web at various different locations in the fabrication process. For instance, the textured web can be formed on the forming fabric, on a transfer fabric, on a bonding fabric or on a combination thereof.

In one embodiment, the web can be subjected to compression after being textured. For example, the web can be fed through a nip formed by a pair of calendaring rolls. The calendaring rolls can partially compress the peak areas formed into the web. Through this process, the density of the peak areas increases in relation to the density of the surrounding valley areas.

According to the present invention, various additives can be incorporated into the airlaid fibrous web. Such additives include super-absorbent materials, odor control materials, scented materials, and the like. The additives can be incorporated homogeneously throughout the web or can be applied at selected locations. For example, in one embodiment, the additives can be located in the valley areas of the webs. In a multi-layered product, the additives can also be positioned between the separate layers.

Other features and aspects of the present invention are discussed in greater detail below.

### BRIEF DESCRIPTION OF THE DRAWINGS

A full and enabling disclosure of the present invention, including the best mode thereof to one of ordinary skill in the art, is set forth more particularly in the remainder specification, including reference to the accompanying figures in which:

FIG. 1 is a simplified cross-sectional view of one embodiment of an airlaying apparatus that can be used in the process of the present invention;

FIG. 2 is a simplified side view of a system for forming airlaid non-woven webs in accordance with the present invention;

FIG. 3 is a perspective view with cutaway portions of one embodiment of an airlaid web made in accordance with the present invention; and

FIG. 4 is a perspective view with cutaway portions of another embodiment of an airlaid web made in accordance with the present invention.

Repeated use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended in limiting the broader aspects of the present invention, which broader aspects are embodied in the exemplary construction.

In general, the present invention is directed to highly textured airlaid webs and to various processes for producing the webs. The airlaid webs are formed on a fabric having a three-dimensional topography. During formation of the airlaid web, a sufficient amount of force is placed on the fibers to cause the web to conform to the three-dimensional surface of the fabric, thus producing a web having a highly textured surface. Once formed, the textured web is bonded together making the structure resilient to compression and fluid collapse.

Textured webs produced according to the present invention can have improved absorption and wicking properties. For instance, the texturized webs of the present invention can be formed with density gradients and varying pore sizes within the fiber structure that enhances the ability of the material to absorb and wick away fluids. Further, since the webs are textured, the webs have an increased surface area for contact with fluids.

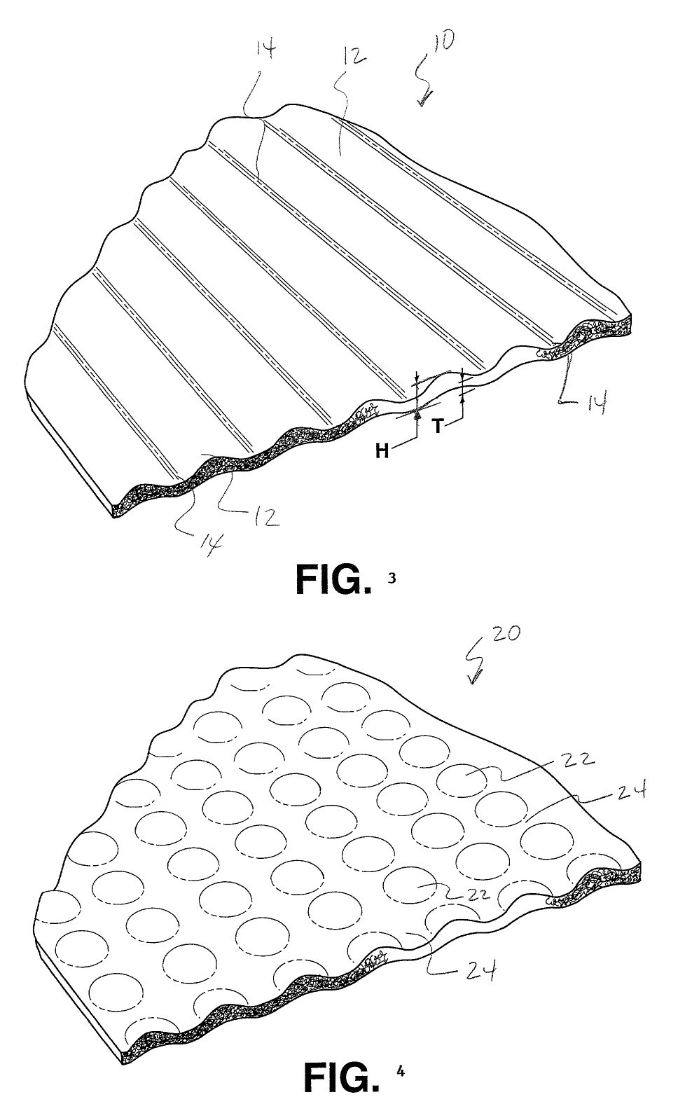

Referring to FIG. 3, for exemplary purposes only, an airlaid fibrous web generally 10 made in accordance with the present invention is shown. As illustrated, the airlaid web 10 includes peak areas 12 separated by valley areas 14. In this embodiment, the peaks and valleys form parallel rows that run in either the machine direction or the cross-machine direction of the web 10. It should be understood, however that the embodiment illustrated in FIG. 3 is merely for explanation purposes only and that any desired textured surface can be made in accordance with the present invention as will be described in more detail below.

In general, airlaid textured webs made according to the present invention will have a repeating pattern of peak areas and valley areas. The peak areas can extend in a single direction as shown in FIG. 3 or can extend in multiple directions over the surface of the web.

For most applications, the textured web of the present invention will contain at least one peak area per inch in one direction of the web, such as in the machine direction or in the cross-machine direction. For example, in some embodiments, the repeating pattern of peak areas and valley areas can include at least 3 peaks areas per inch in one direction, particularly at least 5 peak areas per inch in one direction, more particularly at least 7 peak areas per inch in one direction and in one embodiment, at least 10 peak areas per inch. The amount of peaks present will depend upon the particular application. For instance, the peak areas can be made on a very minute scale such that there are up to 20 peak areas per inch, particularly up to about 32 peak areas per inch, and in one embodiment, up to about 40 peak areas per inch.

When the peak areas extend in multiple directions, the textured webs can include at least 2 peak areas per square inch, particularly at least 9 peak areas per square inch and in some embodiments, at least 15 peak areas per square inch. When extending in multiple directions, it is believed that textured webs can be made having a peak area density of up to about 40 peak areas per square inch or greater.

As shown in FIG. 3, the textured web 10 includes a thickness T or caliper. For most applications, the caliper T will vary throughout the web due to the manner in which the web is formed. In addition to the thickness or caliper, as shown in FIG. 3, the textured web also includes a height H. As used herein, height refers to the distance between the lowest point of the web and the highest point of the web. For example, a web having two textured surfaces will have a height that is equal to the distance from a peak on one surface to a peak on an opposing surface.

For conventionally made flat or planar webs, the height is generally the same distance as the caliper. Texturized webs made according to the present invention, however, have a height H that is much greater than the caliper T of the web. For example, the height of textured webs made according to the present invention can be at least 25% greater than the average caliper of the web. For most applications, the height of the web will be at least 50% greater than the caliper of the web, particularly at least 100% greater than the caliper of the web, and, in one embodiment, 200% greater than the caliper of the web. For patterns where there is a relatively large amount of space between the peak areas, it is also believed that textured webs can be made according to the present invention that have a height that is up to 400% greater than the average caliper of the web.

Because the textured webs of the present invention include a repeating pattern of peak areas and valley areas, the webs have increased surface area in comparison to flat or planar webs and contoured webs. For instance, textured webs made according to the present invention will have a surface area that is at least 25% greater than the surface area of a planar web at the same caliper, particularly greater than 50% of a planar web at the same caliper, more particularly greater than 100% of a planar web at the same caliper, and more particularly greater than 500% of a planar web at the same caliper. In some embodiments, it is believed that a textured web made according to the present invention can have a surface area that is 800% greater than the surface area of a planar web at the same caliper.

Referring to FIG. 4, another embodiment of an airlaid textured web made in accordance with the present invention is illustrated. As shown, the textured web 20 includes peak areas 22 separated by valley areas 24. In this embodiment, the peak areas extend in two directions on the surface of the web. The peak areas also comprise discrete shapes or “islands” that are completely surrounded by the valley areas 24.

The peak areas 22, shown in FIG. 4 appear as small hills. Depending upon the construction of the three-dimensional fabric on which the airlaid web is formed, however, various shapes and designs can be created into the textured surface. For instance, three-dimensional fabrics can be designed that will create airlaid webs having peak areas in particular shapes. The peak areas can be in the shapes of squares, circles, triangles, other aesthetic designs and the like.

In an alternative embodiment, it is also possible to create peak areas 22 that are connected in a reticulated pattern. For example, a three-dimensional forming fabric can be manufactured that will create peak areas that appear in a checkered pattern or in any other desired connected pattern.

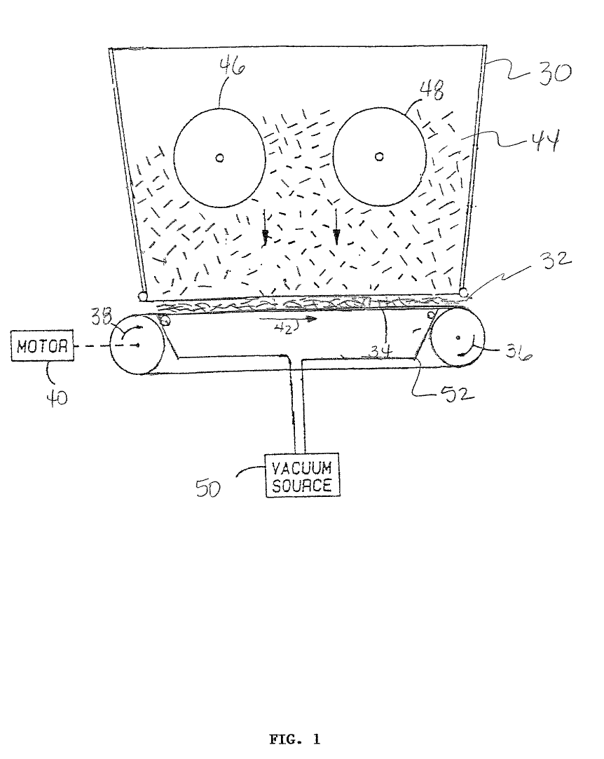

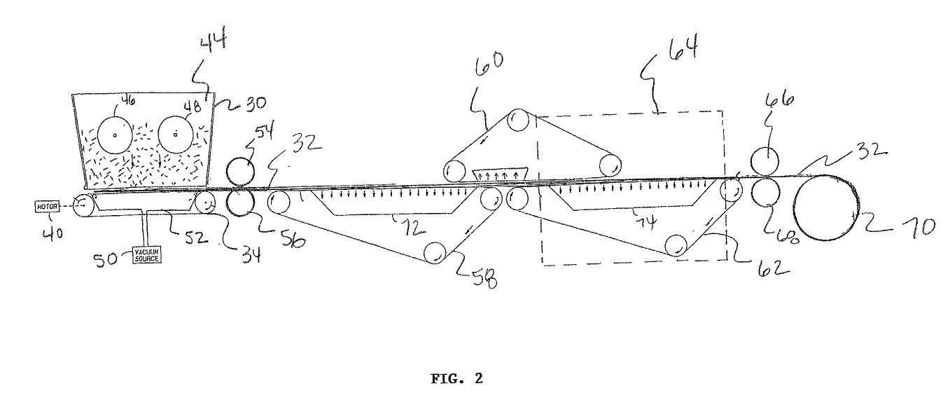

One embodiment of a process for forming textured airlaid webs in accordance with the present invention will now be described in detail with particular reference to FIGS. 1 and 2. Referring to FIG. 1, an airlaying forming station 30 is shown which produces a non-woven web 32 on a forming fabric or screen 34. The forming fabric 34 can be in the form of an endless belt mounted on support rollers 36 and 38. A suitable driving device, such as an electric motor 40 rotates at least one of the support rollers 38 in a direction indicated by the arrows at a selected speed. As a result, the forming fabric 34 moves in a machine direction indicated by the arrow 42.

The forming fabric 34 can be provided in other forms as desired. For example, the forming fabric can be in the form of a circular drum which can be rotated using a motor as disclosed in U.S. Pat. No. 4,666,647, U.S. Pat. No. 4,761,258, or U.S. Pat. No. 6,202,259. The forming fabric 32 can be made of various materials, such as plastic or metal.

As shown, the airlaying forming station 34 includes a forming chamber 44 having end walls and side walls. Within the forming chamber 44 are a pair of material distributors 46 and 48 which distribute fibers and/or other particles inside the forming chamber 44 across the width of the chamber. The material distributors 46 and 48 can be, for instance, rotating cylindrical distributing screens.

In the embodiment shown in FIG. 1, a single forming chamber 44 is illustrated in association with the forming fabric 34. It should be understood, however, that more than one forming chamber can be included in the system. By including multiple forming chambers, layered webs can be formed in which each layer is made from the same or different materials.

Airlaying forming stations as shown in FIG. 1 are available commercially through Dan-Webforming Int. LTD. of Aarhus, Denmark. Other suitable airlaying forming systems are also available from M & J Fibretech of Horsens, Denmark. In general, any suitable airlaying forming system can be used in accordance with the present invention.

As shown in FIG. 1, below the airlaying forming station 30 is a vacuum source 50, such as a conventional blower, for creating a selected pressure differential through the forming chamber 44 to draw the fibrous material against the forming fabric 34. If desired, a blower can also be incorporated into the forming chamber 44 for assisting in blowing the fibers down on to the forming fabric 34.

In one embodiment, the vacuum source 50 is a blower connected to a vacuum box 52 which is located below the forming chamber 44 and the forming fabric 34. The vacuum source 50 creates an airflow indicated by the arrows positioned within the forming chamber 44. Various seals can be used to increase the positive air pressure between the chamber and the forming fabric surface.

During operation, typically a fiber stock is fed to one or more defibrators (not shown) and fed to the material distributors 46 and 48. The material distributors distribute the fibers evenly throughout the forming chamber 44 as shown. Positive airflow created by the vacuum source 50 and possibly an additional blower force the fibers onto the forming fabric 34 thereby forming an airlaid non-woven web 32.

The material that is deposited onto the forming fabric 34 will depend upon the particular application. The fiber material that can be used to form the airlaid web 32, for instance, can include natural fibers, synthetic fibers, and combinations thereof. Examples of natural fibers include wood pulp fibers, cotton fibers, wool fibers, silk fibers and the like, as well as combinations thereof. Wood pulp fibers can include softwood fibers and hardwood fibers. Synthetic fibers can include rayon fibers, polyolefin fibers, polyester fibers and the like, as well as combinations thereof. Polyolefin fibers include polypropylene fibers and polyethylene fibers. The fibers can have various lengths, such as up to about 6 to about 8 millimeters or greater.

In one embodiment, binder fibers can also be incorporated into the airlaid web 32. Once incorporated into the web, the binder fibers can be later heated and at least partially melted and fused together in order to bond the web together. The binder fibers can be, for instance, polyethylene fibers, polypropylene fibers, and polyester fibers. In one embodiment, the binder fibers can be bicomponent fibers that include, for instance, a sheath polymer and a core polymer. During bonding, the sheath polymer melts and bonds to other materials, while the core polymer does not. Suitable bicomponent fibers include polyester/polyethylene fibers, polypropylene/polyethylene fibers, and the like. The bicomponent fibers can be present in the non-woven web in an amount up to 100% by weight, and particularly from about 2% to about 50% by weight depending upon the particular application. In multi-layered products, the amount of bicomponent fibers contained within each layer can also vary. For example, one layer can be made exclusively of bicomponent fibers, while another layer can contain bicomponent fibers in combination with other fibers, such as pulp fibers.

Besides fibers, the airlaid web 32 can include various other additives. For instance, additives such as odor absorbents, antimicrobial agents, super-absorbent materials, scented materials, and the like can be incorporated into the web.

When forming the airlaid web 32 from different materials and fibers, the forming chamber 44 can include multiple inlets for feeding the materials to the chamber. Once in the chamber, the materials can be mixed together if desired. Alternatively, the different materials can be separated into different layers in forming the web. For example, the system can include multiple forming chambers for forming multiple layers of different materials. In one embodiment, a first layer can be made from fibers while a second layer can be made from any of the above-described additives alone or in combination with fibers.

In accordance with the present invention, the airlaid web 32 is formed on a three-dimensional fabric under a sufficient amount of force to cause the web to conform to the surface of the fabric. In this manner, textured webs are produced having peak areas and valley areas as shown in FIGS. 3 and 4. In one embodiment of the present invention, the three dimensional fabric is the forming fabric 34 as shown in FIG. 1. The force that is needed to create the textured web on the three-dimensional forming fabric 34 is created by the vacuum source 50 located under the forming chamber 44 and/or by some other compressive force, e.g. a compaction roll. In general, it is believed that any three-dimensional forming fabric that can impart a repeating pattern of peak areas and valley areas into the web 32 can be used in the process of the present invention. The forming fabric 34, however, must have a permeability that will collect the fibrous material on the surface of the fabric but will allow sufficient air to pass through the fabric in order to generate the force necessary to create a textured surface on the web.

During the above process, a textured web is produced that, on a minute scale, includes peak areas and valley areas that dramatically increase the visible and usable surface area of the web. The resulting structure can be controlled so as to form airlaid webs having improved absorption and wicking properties. In one embodiment, it is believed that the textured webs can also be formed with different density gradients contained within the structure. In particular, it is believed that higher density regions will form in the web at higher localized airflow regions on the forming fabric. For some applications, these higher density areas will be created where the peak areas are formed although the higher density areas can be created in other sections of the web depending on the construction of the forming fabric. In this manner, a textured web is formed having higher density areas capable of absorbing and retaining large amounts of fluids.

In an alternative embodiment, higher density areas can be formed by calendaring the web or otherwise compressing the web after the textured web is formed. If some compression of the web is done after the textured surface is formed, the compression will cause the peak areas to densify and have a higher density than the adjacent valley areas. In one embodiment, the web can be compressed an amount sufficient to form a planar web having high density areas and low density areas.

The difference in density between the peak areas and valley areas will depend upon the manner in which the peaks and valleys are formed. In general, the peak areas can have a density that is at least 25% greater than the density of the valley areas, particularly 50% greater and more particularly 100% greater than the density of the valley areas depending on the desired result.

Referring to FIG. 2, one embodiment of an entire web forming system incorporating the airlaying station 30 of FIG. 1 is shown. In this embodiment, the web 32 is formed under the airlaying forming station 30 and fed between a pair of calendaring rolls 54 and 56. The calendaring rolls 54 and 56 are optional and provided for exemplary purposes only. The calendaring rolls 54 and 56 can be used to increase the density of the peak areas as described above.

From the calendaring rolls 54 and 56, the airlaid web 32 is supported on a transfer fabric 58. A second transfer fabric 60 also appears in this embodiment for facilitating movement of the web from the transfer fabric 58 to a bonding fabric 62. As shown in phantom, the bonding fabric 62 is placed in association with a bonding station 64. The bonding station 64 can be an oven if the airlaid fabric contains binder fibers or can be an adhesive application station.

From the bonding fabric 62, in this embodiment, a further pair of calendaring rolls 66 and 68 are shown which form a nip through which the airlaid web is fed. From the calendaring rolls 66 and 68, the airlaid web 32 is wound onto a reel 70. Similar to calendaring rolls 54 and 56, calendaring rolls 66 and 68 are optional, but can be provided in the system in order to alter the properties and characteristics of the textured web.

As described above, the textured webs of the present invention are formed on a three-dimensional fabric that imparts a textured surface to the airlaid web. In one embodiment as described above, the three-dimensional fabric can be the forming fabric 34 as shown in FIG. 1. It should be understood, however, that the three-dimensional fabric used to form the textured surface can be contained in any other portion of the web-forming system. For example, in an alternative embodiment, the three-dimensional fabric can be the transfer fabric 58 shown in FIG. 2. As shown, when it is desired to impart a textured surface into an airlaid web, the transfer fabric 58 can be placed in association with a vacuum box 72. Vacuum box 72 provides the force sufficient to draw the airlaid web 32 onto the three-dimensional fabric 58 and form the textured surface. In other embodiments, a compression roller can also be used alone or in combination with the vacuum box.

In still another alternative embodiment, the three-dimensional fabric can be the bonding fabric 62. As shown, the bonding fabric 62 can be placed in association with a vacuum box 74 (and/or another compression device) for creating the textured impressions in the web.

During the above-described process, webs can be produced having a textured surface. Depending upon the process conditions and the materials that are used to make the web, the web can be formed having a textured surface only on one side or can be formed having two textured surfaces. For example, in some embodiments, the textured surface that is formed on one side of the airlaid web will carry through to the opposite side of the web, depending upon the amount of force applied to the web during formation of the textured surface and the basis weight of the web.

In an alternative embodiment, the process of the present invention can produce a web having a textured surface on one side and a relatively flat and planar surface on the opposite surface. Further, multi-layered webs can be formed with a single textured surface. In this embodiment, a first layer can be formed with a textured surface. After the textured surface is created, further layers can be deposited on one side of the web to form a composite structure having a textured side and a planar side.

In still another alternative embodiment of the present invention, the web can be formed with a textured surface as described above. The textured surface can be formed on the forming fabric, a transfer fabric or a bonding fabric. Once one side of the web has been pressed against a three dimensional fabric to form the textured surface, the opposite side of the web can then be pressed against a three dimensional fabric for forming a textured surface on the opposite side of the web. The textured pattern formed into the first side of the web and the opposite side of the web can be the same or different depending upon the three dimensional fabrics used to produce the web. In this embodiment, the web can have a single layer or a multi-layer construction.

After or during formation of the textured surface, the airlaid web 32 is bonded together. By bonding the structure together, the structure becomes resilient to compression and fluid collapse. As described above, the web 32 can be bonded thermally by using an oven 64. During thermal bonding, a binder material, such as binder fibers, contained in the non-woven web is melted. Upon solidification, the binder material contained in the web bonds the material together.

Alternatively, however, the bonding station 64 can be an adhesive application station. The adhesive application station can be used alone or in combination with thermal bonding. In this embodiment, an adhesive is applied to the surfaces of the web for bonding the web together. The adhesive can be applied to the web through spraying, printing such as rotogravure printing or flexographic printing, or by any other suitable process. The adhesive can be, for instance, an ethylene vinyl acetate copolymer adhesive, a starch adhesive, an acrylic adhesive, a polyvinyl alcohol adhesive or any other suitable adhesive.

In one embodiment, the formed airlaid web 32 besides being textured, can be contoured for a particular application. The web can be contoured by being placed in a mold under pressure or, alternatively, by using a scarfing apparatus as disclosed in U.S. Pat. No. 4,626,184 which is incorporated herein by reference.

The web can include a contour for various reasons. For example a contour can be built into the web for making the web better suited for use with absorbent articles, such as feminine hygiene products. In addition, making the web contoured can facilitate absorption of fluids. For example, in one embodiment, the web can be molded such that the outer edges of the web are raised in comparison to the middle of the web. In this manner, a cup-like shape is formed that better traps fluids, especially high viscous fluids.

The basis weight of webs made according to the present invention can vary dramatically depending upon the particular application. For many applications, the textured airlaid web will have a basis weight of at least 40 gsm, and particularly from 50 gsm to about 1500 gsm or higher. In one embodiment, the basis weight of the web can be from about 50 gsm to about 700 gsm.

The overall density of the web can also vary depending upon the materials used to form the web, the process used to form the web, and the desired result. In general the density of the web can be from about 0.01 grams per cubic centimeter to about 0.3 grams per cubic centimeter.

Textured webs made according to the present invention can be used in a limitless variety of different absorbent articles and products. For instance, the airlaid textured webs can be incorporated into feminine hygiene products, wipers, adult incontinent products, diapers, and the like. Once incorporated into an absorbent product, the textured web can serve as the outer lining, as a surge layer, or as an absorbent layer.

These and other modifications and variations to the present invention may be understood and realized by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A textured airlaid fibrous web comprising natural fibers, synthetic fibers, or mixtures thereof, said airlaid web being formed on a three-dimensional fabric under sufficient force to cause the web to conform to the surface of the fabric, the textured web including a repeating pattern of peaks and valleys, the textured web having a height that is at least 25% greater than the average caliper of the web, the airlaid web being bonded together.

2. A textured airlaid web as defined in claim 1, wherein the airlaid web is thermally bonded together.

3. A textured airlaid web as defined in claim 2, wherein the web contains binder fibers that thermally bond the web together.

4. A textured airlaid web as defined in claim 1, wherein the web is bonded together by applying an adhesive to the surfaces of the web.

5. A textured airlaid web as defined in claim 1, wherein the web is bonded together thermally and through the use of an adhesive.

6. A textured airlaid web as defined in claim 1, wherein the peaks contained within the repeating pattern have a higher density than the valleys.

7. A textured airlaid web as defined in claim 6, wherein the density of the peaks is at least 25% greater than the density of the valleys.

8. A textured airlaid web as defined in claim 1, wherein the web has a basis weight of from about 40 gsm to about 1500 gsm.

9. A textured airlaid web as defined in claim 1, wherein the web includes at least 1 peak per inch in one direction of the web.

10. A textured airlaid web as defined in claim 1, wherein the web includes at least 5 peaks per inch in one direction of the web.

11. A textured airlaid web as defined in claim 1, wherein the web includes at least 10 peaks per inch in one direction of the web.

12. A textured airlaid web as defined in claim 1, wherein the peaks are present in at least two directions on the web, the peaks being present in an amount of at least 2 peaks per square inch.

13. A textured airlaid web as defined in claim 12, wherein the peaks are present in an amount of at least 9 peaks per square inch.

14. A textured airlaid web as defined in claim 1, wherein the web has a surface area that is at least 50% greater than the surface area of a planar web made from the same fibers at the same basis weight.

15. A textured airlaid web as defined in claim 1, wherein the web has a surface area that is at least 100% greater than the surface area of a planar web made from the same fibers at the same basis weight.

16. A textured airlaid web as defined in claim 1, wherein the web is further contoured to have a preselected overall shape.

17. A textured airlaid web as defined in claim 1, wherein the web includes a first surface and a second and opposite surface, the first surface defining the pattern of peaks and valleys, the second surface comprising a substantially planar surface.

18. A textured airlaid web as defined in claim 1, wherein the web includes a first surface and a second surface, the first surface including a first pattern of peaks and valleys, the second surface including a second pattern of peaks and valleys.

19. A textured airlaid web as defined in claim 1, wherein the airlaid web is multi-layered.

20. A textured airlaid web as defined in claim 1, wherein the textured web has a height that is at least 50% greater than the average caliper of the web.

21. A medical absorbent product containing the textured airlaid web as defined in claim 1.

22. A diaper containing the textured airlaid web as defined in claim 1.

23. A feminine hygiene product containing the textured airlaid web as defined in claim 1.

24. An adult incontinence product containing the textured airlaid web as defined in claim 1.

25. A wiper product containing the textured airlaid web as defined in claim 1.

26. A training pant containing the textured airlaid web as defined in claim 1.

27. A textured airlaid web as defined in claim 1, further containing an additive selected from the group consisting of a super-absorbent material, an odor absorbent material, a scented material, an anti-microbial agent, and mixtures thereof.

28. An airlaid fibrous web comprising at least one textured surface, the textured surface including peak areas and valley areas, the peak areas and the valley areas forming a repeating pattern on the surface of the web, the airlaid web having a height that is at least 25% greater than the average caliper of the web, the web including at least one peak area per inch in one direction of the web, the airlaid web being bonded together.

29. An airlaid fibrous web as defined in claim 28, wherein the textured surface is formed on a fabric having a three-dimensional surface under sufficient force to cause the web to conform to the three-dimensional surface of the fabric.

30. An airlaid fibrous web as defined in claim 28, wherein the airlaid web is thermally bonded together.

31. An airlaid fibrous web as defined in claim 28, wherein the web is bonded together by applying an adhesive to the surfaces of the web.

32. An airlaid fibrous web as defined in claim 28, wherein the peak areas have a first density and the valley areas have a second density, the first density being at least 25% greater than the second density.

33. An airlaid fibrous web as defined in claim 28, wherein the web includes at least 3 peak areas per inch in one direction of the web.

34. An airlaid fibrous web as defined in claim 28, wherein the web includes at least 5 peak areas per inch in one direction of the web.

35. An airlaid fibrous web as defined in claim 28, wherein the peak areas are present in at least two directions on the web, the peak areas being present in an amount of at least 2 peaks per square inch.

36. An airlaid fibrous web as defined in claim 28, wherein the peak areas are present in at least two directions on the web, the peak areas being present in an amount of at least 9 peaks per square inch.

37. An airlaid fibrous web as defined in claim 28, wherein the textured surface has a surface area that is at least 50% greater than the surface area of a planar web having the same basis weight.

38. An airlaid fibrous web as defined in claim 28, wherein the textured surface has a surface area that is at least 200% greater than the surface area of a planar web having the same basis weight.

39. An airlaid fibrous web as defined in claim 28, further containing an additive selected from the group consisting of a super-absorbent material, an odor absorbent material, a scented material, an anti-microbial agent, and mixtures thereof.

40. An airlaid fibrous web as defined in claim 28, wherein the web includes a substantially planar surface opposite the textured surface.

41. An airlaid fibrous web as defined in claim 28, wherein the airlaid web has a height that is at least 50% greater than the average caliper of the web.

42. An airlaid fibrous web as defined in claim 28, wherein the airlaid web has a height that is at least 100% greater than the average caliper of the web.

43. An airlaid fibrous web as defined in claim 28, wherein the textured surface has a surface area that is at least 400% greater than the surface area of a planar web having the same basis weight.

44. An airlaid fibrous web as defined in claim 28, wherein the airlaid web has a height that is at least 100% greater than the average caliper of the web.

45. An airlaid fibrous web as defined in claim 28, wherein the web consists essentially of synthetic fibers.

46. An airlaid fibrous web as defined in claim 28, wherein the peak areas have a first density and the valley areas have a second density, the first density being at least 100% greater than the second density.

47. A textured airlaid fibrous web comprising natural fibers, synthetic fibers, or mixtures thereof, the airlaid web being formed on a three dimensional fabric under sufficient force to cause the web to conform to the surface of the fabric, the web including a textured surface having a repeating pattern of peak areas and valley areas, the airlaid web having a height that is at least 25% greater than the average caliper of the web, the web including at least 5 peak areas per inch in one direction of the web and including at least 9 peak areas per square inch, the airlaid web being bonded together, the airlaid web having a basis weight of at least 40 gsm.
